(19) United States
(12) Patent Application Publication
(10) Pub. No.: US 2026/0241062 A1
(43) Pub. Date: Aug. 20, 2026

(54) WATER-DISPERSED PRESSURE-SENSITIVE ADHESIVE COMPOSITION AND PRESSURE-SENSITIVE ADHESIVE SHEET

(71) Applicant: NITTO DENKO CORPORATION, Ibaraki-shi, Osaka (JP)

(72) Inventor: Keisuke HIRANO, Ibaraki-shi, Osaka (JP)

(21) Appl. No.: 19/161,014

(22) PCT Filed: Feb. 5, 2024
(86) PCT No.: PCT/JP2024/003748

§ 371 Date: Aug. 29, 2025

(30) Foreign Application Priority Data
Mar. 2, 2023 (JP) 2023-031657

(51) Int. Cl. A61L 24/06; C08F 220/18

(52) U.S. Cl. CPC A61L 24/06; C08F 220/1808

(57) ABSTRACT

Provided is a water-dispersed pressure-sensitive adhesive composition comprising an acrylic polymer. The acrylic polymer is a polymer of a monomer mixture that includes an alkyl (meth)acrylate (m1) as the primary component and further includes a carboxy group-containing monomer (m2) and an N-containing but NH-free monomer (m3). The carboxy group-containing monomer (m2) includes acrylic acid (m2a) and a monomer (m2b) that is more hydrophobic than acrylic acid (m2a).

### TECHNICAL FIELD

The present invention relates to a water-dispersed pressure-sensitive adhesive composition and a pressure-sensitive adhesive sheet.

The present application claims priority based on Japanese Patent Application No. 2023-031657 filed on Mar. 2, 2023; and the entire contents thereof are incorporated herein by reference.

### BACKGROUND ART

In general, pressure-sensitive adhesive (PSA) exists as a soft solid (a viscoelastic material) in a room temperature range and has a property to adhere easily to an adherend with some pressure applied. For such a property, PSA has been widely used in various fields as a supported PSA sheet having a PSA layer on a support or as a support-less PSA sheet free of a support. Technical documents about PSA sheets include Patent Documents 1 to 3. Patent Documents 1 to 3 teach a PSA applied to the skin (also called a dermal PSA).

### CITATION LIST

### Patent Literature

[Patent Document 1] Japanese Patent Application Publication No. 2006-297046
[Patent Document 2] Japanese Patent Application Publication No. 2011-166
[Patent Document 3] Japanese Patent Application Publication No. 2019-47961

### SUMMARY OF INVENTION

### Technical Problem

PSA sheets may need to have various properties depending on how they are used. For instance, dermal PSAs used in medical applications are required not to cause rashes or itchiness on the adherend skin. The skin surface has skin furrows (fine grooves in a net-like pattern) and skin ridges (hills surrounded by the skin furrows) with follicles located at intersections of the skin furrows and sweat pores centrally located in the skin ridges. Thus, the skin surface has a texture pattern formed by fine recessed lines. Skin surface conditions vary greatly from person to person, and even for the same person, they differ depending on application areas. The skin surface and its texture stretch and deform due to movements, etc., and further change depending on the state of dryness and sweating. Dermal PSAs need to adhere firmly to such various surface conditions. In particular, PSA applied to the skin has a unique problem in that sweating on the adherend skin generates moisture at the adhesive interface with the PSA, loosening the adhesion and making the PSA more susceptible to peeling. Dermal PSA must have the ability to maintain tight adhesion to the skin even when sweating (sweat resistance).

Regarding measures against sweat for dermal PSA, for instance, the PSA surface is provided with depressions serving as sweat flow channels to eliminate the sweat from the PSA application area in a known method (e.g., partial application in Patent Document 1). However, such a method reduces the PSA application area, inevitably loosening the adhesion. Patent Document 2 studies a method of providing moisture permeability to a PSA by adding sodium polyacrylate as a water-absorbing polymer to the PSA tape. This method is effective when the PSA has a low moisture content. However, when the moisture content (the amount of water retained) increases due to sweating, etc., the water absorption by the sodium polyacrylate causes a rapid decrease in adhesive strength. Patent Document 3 considers a method of obtaining high moisture permeability by incorporating thermally expandable particles into a PSA and foaming them when the PSA is applied, thereby forming an uneven texture on the PSA surface. However, to maintain the uneven texture on the PSA surface, the PSA to be used needs to have high glass transition temperature and gel fraction, making this method available only for weak-adhesive applications such as removable applications.

Recently, due to environmental considerations, there has been a trend towards eliminating solvents in PSAs, promoting the use of solvent-free PSAs such as water-dispersed PSAs in various applications. However, water-dispersed PSAs generally tend to have lower water resistance than solvent-based PSAs made using organic solvents. This, for instance, makes them disadvantageous for use as dermal PSAs requiring sweat resistance as described above. In particular, the emulsifier in water-dispersed PSAs helps water penetrate into the PSA. This is why their adhesive strength tend to decrease upon contact with water. As emulsifiers have a lower molecular weight in general, after the PSA is formed, they migrate to the PSA surface, that is, to the interface with the skin, likely loosening adhesion and reducing sweat resistance.

In a water-dispersed PSA, one possible method for improving the water resistance (especially the sweat resistance) is to reduce the water absorption of the PSA itself, for instance, by not using hydrophilization factors (i.e., emulsifiers and monomers having a polar functional group), or by limiting their amount used. However, in practice, it is extremely difficult to prepare water-dispersed PSAs without using emulsifiers. In a water-dispersed PSA composition, fine particles of adhesive polymer are dispersed in water. Good particle dispersion depends on the charge repulsion of the functional groups (typically carboxy groups) on the particle surface that are introduced by copolymerization of polar functional group-containing monomers such as acrylic acid. Thus, the amount of polar functional group-containing monomer used can only be reduced to the extent that it does not impair the dispersion stability of the particles in the dispersion. As such, there are restrictions on manufacturing and other aspects in improving the water resistance of water-dispersible PSAs.

It is thought that adhesion to the skin can be improved by tightening the interfacial adhesion and increasing the deformation energy (bulk cohesive strength) of the PSA. The interfacial adhesion can be tightened by using a highly flexible PSA that can penetrate into the skin surface texture, thereby increasing the contact area with the skin interface. On the other hand, in particular, the deformation energy of the PSA is associated with stringing deformation of the PSA during its removal from the skin. An increase in deformation energy consumption can enhance the adhesion to the skin. Specifically, by increasing the cohesive strength through copolymerization of a high Tg monomer (a monomer whose homopolymer has a high glass transition temperature) or a polar functional group-containing monomer, the amount of energy consumed during the PSA (bulk) deformation can be increased. However, such a design reduces the PSA flexibility and thus the interfacial adhesion, making it difficult to use as a dermal PSA. In short, there is a trade-off between the tightness of interfacial adhesion and deformation energy of a PSA, and it is not easy to combine both and obtain tighter adhesion to the skin.

The present invention has been made in view of these circumstances with an objective to provide a water-dispersed PSA composition capable of forming a PSA that has high adhesive strength and good water resistance even when designed to be flexible. Another related purpose is to provide a PSA sheet having a PSA layer formed using the water-dispersed PSA composition.

### Solution to Problem

This description provides a water-dispersed PSA composition comprising an acrylic polymer. The acrylic polymer is a polymer of a monomer mixture that comprises an alkyl (meth)acrylate (m1) as the primary component and further comprises a carboxy group-containing monomer (m2) and a nitrogen-atom-containing (N-containing) monomer (m3) with no hydrogen atoms bonded to the nitrogen atom. The carboxy group-containing monomer (m2) includes acrylic acid (m2a) and a monomer (m2b) that is more hydrophobic than acrylic acid (m2a). Acrylic-polymer-containing water-dispersed PSA compositions with such monomer mixture can form a PSA with high adhesive strength and good water resistance, even when designed to be flexible.

In some preferable embodiments, a possible N-containing monomer (m3) has an acid dissociation constant (pKa) in the range of −2 or higher and 1 or lower. The N-containing monomer (m3) preferably brings about the effect of the art disclosed herein.

In some preferable embodiments, as the N-containing monomer (m3), an N-containing cyclic monomer having a cyclic nitrogen atom (or “N-heterocyclic monomer” hereinafter) is used. The N-containing monomer (m3) can realize superior adhesive strength and water resistance.

In some preferable embodiments, as the monomer (m2b) more hydrophobic than acrylic acid, a carboxy group-containing monomer having a water solubility in the range of 2.0 g/100 mL to 15.0 g/100 mL at 20° C. is used. The combined use of acrylic acid (m2a) and such a suitably hydrophobic monomer (m2b) preferably bring about the effect of the art disclosed herein.

In some preferable embodiments, the monomer mixture has 0.5 wt % (% by weight) to 5 wt % carboxy group-containing monomer (m2) and 0.1 wt % to 5 wt % N-containing monomer (m3). By using the carboxyl group-containing monomer (m2) and the N-containing monomer (m3) in amounts within such limited ranges, good flexibility and water resistance can be preferably obtained with high adhesive strength.

In some embodiments, the monomer mixture further comprises a silane-based monomer. In the water-dispersed PSA, the acrylic polymer with a silane-based monomer unit can increase the cohesive strength through crosslinking of polymer particles, leading to greater water resistance. This description also provides a PSA sheet. The PSA sheet has a PSA layer formed from a water-dispersed PSA composition disclosed herein. The PSA sheet can have high adhesive strength and good water resistance even when designed to be flexible.

The PSA sheet disclosed herein has high adhesive strength and good water resistance even with a flexible design. Thus, based on the flexibility, adhesive strength and water resistance, it can be preferably used as a dermal PSA sheet that tightly adheres to the skin and have good sweat resistance.

### BRIEF DESCRIPTION OF DRAWINGS

FIG. 1 shows a cross-sectional diagram schematically illustrating the PSA sheet according to an embodiment.

### DESCRIPTION OF EMBODIMENTS

Preferable embodiments of the present invention are described below. Matters necessary to practice this invention other than those specifically referred to in this Description can be understood by a person skilled in the art based on the disclosure about implementing the invention in this Description and common technical knowledge at the time the application was filed. The present invention can be practiced based on the contents disclosed in this Description and common technical knowledge in the subject field. In the following drawings, components or units having the same functions may be described with the same symbols allocated and redundant description may be omitted or simplified. The embodiments illustrated in the drawings are schematic in order to clearly describe the present invention and the drawings do not accurately represent the size or scale of products actually provided.

As used herein, the term “PSA” refers to, as described earlier, a material that exists as soft solid (a viscoelastic material) in a room temperature range and has a property to adhere easily to an adherend with some pressure applied. As defined in “Adhesion: Fundamentals and Practice” by C. A. Dahlquist, McLaren & Sons (1966), P. 143), the PSA referred to herein can be a material that has a property satisfying complex tensile modulus E*(1 Hz)<10⁷ dyne/cm² (typically, a material that exhibits the described characteristics at 25° C.).

### <Water-Dispersed PSA Composition>

The PSA composition disclosed herein is a water-dispersed PSA composition in which at least a portion of the components is dispersed in an aqueous medium. The water-dispersed form includes a suspended state and an emulsified state. The aqueous medium here refers to a medium formed of a solvent, the solvent being water or a mixed solvent (aqueous solvent) whose primary component is water. The concept of water-dispersed PSA composition encompasses a so-called emulsion-based PSA.

### (Acrylic Polymer)

The PSA composition disclosed herein comprises an acrylic polymer. The acrylic polymer is typically included as the base polymer in the PSA composition. The “base polymer” here refers to the primary component among the polymers in the PSA composition (which can also be a PSA) and is not to be construed as being limited to anything other than this. As used herein, the “primary component” refers to the highest-content (most abundant) component by weight among the components included. Thus, for instance, when the PSA composition comprises three or more kinds of polymers, the amount of primary component among the polymers is 34% by weight (wt %) or greater. The component may typically account for more than 50 wt %. The water-dispersed PSA composition disclosed herein typically has an emulsion form with the acrylic polymer dispersed in water.

As used herein, the term “acrylic polymer” refers to a polymer derived from a monomer mixture including more than 50% acrylic monomer by weight, or an acrylic polymer. The acrylic monomer refers to a monomer having at least one (meth)acryloyl group per molecule. As used herein, the term “(meth)acryloyl” comprehensively refers to acryloyl and methacryloyl. Similarly, the term “(meth)acrylate” comprehensively refers to acrylate and methacrylate, and the term “(meth)acryl” comprehensively refers to acryl and methacryl.

As the acrylic resin disclosed herein, a polymer of a monomer mixture comprising an alkyl (meth)acrylate (m1) as the primary component is used. For instance, a resin comprises, as the base polymer, an acrylic polymer formed from monomers (or the monomer mixture) including an alkyl (meth)acrylate having a linear or branched alkyl group with 1 up to 20 carbon atoms at the ester terminus. Hereinafter, an alkyl (meth)acrylate having, at the ester terminus, an alkyl group with X up to Y number of carbon atoms may be referred to as an “C_{X-Y} alkyl (meth)acrylate.” In some preferable embodiments, in view of obtaining desirable adhesive properties (adhesive strength, cohesive strength, etc.), the amount of alkyl (meth)acrylate (m1) in all monomers of the acrylic polymer is suitably above 50% by weight, preferably 70% by weight or higher, more preferably 80% by weight or higher, yet more preferably 90% by weight or higher, for instance, 92% by weight or lower, or even 94% by weight or lower. In view of obtaining the undermentioned effects based on copolymerization of a carboxy group-containing monomer (m2) and an N-containing monomer (m3), the alkyl (meth)acrylate content of the monomer mixture is, for instance, 99.4 wt % or lower, preferably 99 wt % lower, more preferably 98 wt % or lower, or yet more preferably 97 wt % or lower. For the alkyl (meth)acrylate, solely one species or a combination of two or more species can be used.

Non-limiting specific examples of the C₁₋₂₀ alkyl (meth)acrylate include methyl (meth)acrylate, ethyl (meth)acrylate, propyl (meth)acrylate, isopropyl (meth)acrylate, n-butyl (meth)acrylate, isobutyl (meth)acrylate, s-butyl (meth)acrylate, t-butyl (meth)acrylate, pentyl (meth)acrylate, isopentyl (meth)acrylate, hexyl (meth)acrylate, heptyl (meth)acrylate, octyl (meth)acrylate, 2-ethylhexyl (meth)acrylate, isooctyl (meth)acrylate, nonyl (meth)acrylate, isononyl (meth)acrylate, decyl (meth)acrylate, isodecyl (meth)acrylate, undecyl (meth)acrylate, dodecyl (meth)acrylate, tridecyl (meth)acrylate, tetradecyl (meth)acrylate, pentadecyl (meth)acrylate, hexadecyl (meth)acrylate, heptadecyl (meth)acrylate, stearyl (meth)acrylate, isostearyl (meth)acrylate, nonadecyl (meth)acrylate, and eicosyl (meth)acrylate.

In some embodiments, among the C₁₋₂₀ alkyl (meth)acrylates, it is preferable to use at least a C₄₋₂₀ alkyl (meth)acrylate, more preferable to use a C₄₋₁₈ alkyl (meth)acrylate, yet more preferable to use a C₄₋₁₂ alkyl (meth)acrylate, or particularly preferable to use a C₄₋₁₀ alkyl (meth)acrylate. In some embodiments, in the monomers forming the acrylic polymer, the amount of C₄₋₁₈ alkyl (meth)acrylate (preferably C₄₋₁₂, or more preferably C₄₋₁₀ alkyl (meth)acrylate) in C₁₋₂₀ alkyl (meth)acrylate is preferably 50 wt % or higher, more preferably 70 wt % or higher, yet more preferably 90 wt % or higher, or may be 95 wt % or higher, or 98 wt % to 100 wt %. The use of such an alkyl (meta) acrylate as the primary component helps obtain good adhesive properties.

In some preferable embodiments, as the C₁₋₂₀ alkyl (meth)acrylate, a C₇₋₉ alkyl acrylate is used. The use of C₇₋₉ alkyl acrylate can preferably bring about a PSA having good adhesive strength and excellent flexibility. In some preferable embodiments, in the monomers forming the acrylic polymer, the C₇₋₉ alkyl acrylate content of the C₁₋₂₀ alkyl (meth)acrylate is, for instance, suitably 50 wt % or higher, more preferably 70 wt % or higher, yet more preferably 90 wt % or higher, or may be 95 wt % or higher, or 98 wt % to 100 wt %. For instance, 2-ethylhexyl acrylate (2EHA) is particularly preferable as the monomer. Other examples of preferable C₇₋₉ alkyl acrylates include heptyl acrylate, octyl acrylate, isooctyl acrylate and isononyl acrylate.

The monomer mixture forming the acrylic polymer include a carboxy group-containing monomer (m2) and an N-containing monomer (m3) with no hydrogen atoms bonded to the nitrogen atom (or “N-containing but NH-free monomer” hereinafter). The combined use of the monomers (m2 and m3) can increase adhesive strength and water resistance. This can be that in the copresence of the carboxy group-containing monomer (m2) and N-containing monomer (m3), their acid-base interactions yield an increase in cohesive strength, with the cohesive strength contributing to greater adhesive strength and water resistance. It is noted that the art disclosed herein is not limited to the above considerations. By taking advantage of the combined effect of the carboxy group-containing monomer (m2) and N-containing monomer (m3), for instance, a highly flexible material can be used as the primary monomer, alkyl (meta) acrylate (m1), to combine flexibility, adhesive strength and water resistance.

The carboxy group-containing monomer (m2) used as an aforementioned monomer includes acrylic acid (m2a) and a monomer (m2b) that is more hydrophobic than acrylic acid (m2a). The combined use of acrylic acid (m2a) and a relatively hydrophobic carboxy group-containing monomer (m2b) as the carboxy group-containing monomer (m2) can increase adhesive strength and water resistance. One possible reason for this is that in synthesizing the water-dispersed acrylic polymer, the relatively polar acrylic acid (m2a) is present on the surface of the polymer particles in the dispersion and interacts with the N-containing monomer (m3) to contribute to the dispersion stability of the particles and the ease of preparing the dispersion, whereas much of the relatively hydrophobic carboxyl group-containing monomer (m2b) is present inside the polymer particles due to its hydrophobicity and increase the internal cohesive strength of the polymer particles based on acid-base interactions with the N-containing monomer (m3). It is thought that as a result, even after the polymer particles are fused together by subsequent drying to form the PSA, the PSA has high cohesive strength as a bulk, which in turn increases adhesive strength and water resistance. It is noted that the art disclosed herein is not limited to the above considerations. Whether or not the carboxy group-containing monomer (m2b) is more hydrophobic than acrylic acid can be determined from its chemical structure, and also from its water solubility at 20° C. described later.

Examples of the monomer (m2b) more hydrophobic than acrylic acid (m2a) include ethylenically unsaturated monocarboxylic acids such as methacrylic acid (MAA), carboxyethyl (meth)acrylate, carboxypentyl (meth)acrylate, 2-(meth)acryloyloxyethyl hexahydrophthalic acid, 2-(meth)acryloyloxypropyl hexahydrophthalic acid, 2-(meth)acryloyloxyethyl phthalic acid, 2-(meth)acryloyloxyethyl succinic acid, 2-(meth)acryloyloxyethyl-2-hydroxyethylphthalic acid, 2-(meth)acryloyloxyethyl maleic acid, carboxypolycaprolactone mono(meth)acrylate, 2-(meth)acryloyloxyethyl tetrahydrophthalic acid, crotonic acid and isocrotonic acid. For the monomer (m2b), solely one species or a combination of two or more species can be used. Among them, MAA is preferable for being effective in small amounts due to its low molecular weight.

In some preferable embodiments, as the carboxy group-containing monomer (m2b) more hydrophobic than acrylic acid, a monomer having a water solubility (solubility in water) at 20° C. of 15.0 g/100 mL or less is used. By using both acrylic acid and a hydrophobic carboxy group-containing monomer (m2b) with water solubility at or below the prescribed value, the monomer (m2b) exists in a suitable amount inside the polymer particles, contributing to increasing the internal cohesive strength of the particles. In view of increasing the internal cohesive strength of polymer particles, the water solubility of the carboxy group-containing monomer (m2b) is preferably 12.0 g/100 mL or less, or may be 10.0 g/100 mL or less, 8.0 g/100 mL or less, or 5.0 g/100 mL or less. In view of ease of emulsion polymerization, the water solubility of the carboxy group-containing monomer (m2b) is preferably 2.0 g/100 mL or greater, or may be 6.0 g/100 mL or greater, or 8.0 g/100 mL or greater. As the water solubility of a monomer mixture at 20° C., it is possible to use a value determined based on Techniques of Chemistry 4th Ed. Vol. II Organic Solvents (1985): New York, NY. John Wiley and Sons, Inc. ICSC(J) 1997. The same applies to the working examples described later.

The ratio of acrylic acid (m2a) to the carboxy group-containing monomer (m2b) more hydrophobic than acrylic acid is not particularly limited. A suitable ratio can be adopted according to the dispersion stability and ease of polymer preparation, the species of carboxy group-containing monomer (m2b) used, etc. In some embodiments, in view of dispersion stability, ease of dispersion preparation, etc., the weight ratio (m2a/m2b) of acrylic acid (m2a) to carboxy group-containing monomer (m2b) more hydrophobic than acrylic acid can be, for instance, 0.1 or higher, 0.5 or higher, 1 or higher (e.g., above 1), 1.5 or higher, or 1.8 or higher. In some embodiments, in view of increasing the adhesive strength and water resistance, the ratio (m2a/m2b) can be, for instance, 10 or lower, 5 or lower, 3 or lower, or 2 or lower.

While no particular limitations are imposed, in some embodiments, the amount of acrylic acid (m2a) used (i.e., the copolymerization ratio of acrylic acid (m2a) in the acrylic polymer) may be, for instance, 0.1 wt % or more of the entire monomer mixture, is preferably 0.5 wt % or more, or more preferably 1.0 wt % or more (e.g., 1.5 wt % or more). By using at least such a prescribed amount of acrylic acid, it is possible to improve adhesive properties (adhesive strength, cohesive strength, etc.), dispersion stability, ease of dispersion preparation, etc. In some embodiments, in view of water resistance, flexibility, etc., the amount of acrylic acid (m2a) is, for instance, suitably less than 10 wt % of the entire monomer mixture, preferably less than 5.0 wt %, or more preferably less than 3.0 wt %.

While no particular limitations are imposed, in some embodiments, the amount of carboxy group-containing monomer (m2b) more hydrophobic than acrylic acid (i.e., the copolymerization ratio of carboxy group-containing monomer (m2b) in acrylic polymer) may be, for instance, 0.1 wt % or more of the entire monomer mixture, is preferably 0.3 wt % or more, more preferably 0.7 wt % or more, or may be more than 1.0 wt % (e.g., 1.5 wt % or more). By increasing the amount of carboxy group-containing monomer (m2b) within the prescribed ranges, the adhesive strength, cohesive strength and water resistance can be increased. In some embodiments, in view of water resistance, flexibility, etc., the amount of carboxy group-containing monomer (m2b) used is, for instance, suitably less than 10 wt % of the entire monomer mixture, preferably less than 5.0 wt %, or more preferably less than 3.0 wt %, for instance, may be 1.5 wt % or less.

The amount of carboxy group-containing monomer (m2) (i.e., the copolymerization ratio of carboxy group-containing monomer (m2) in acrylic polymer) is not particularly limited. It can be, for instance, 0.1 wt % or more of the entire monomer mixture, preferably 0.5 wt % or more, more preferably 1.0 wt % or more, yet more preferably 2.0 wt % or more, particularly preferably 2.5 wt % or more, or may be 3.0 wt % or more (e.g., 3.5 wt % or more). By increasing the amount of carboxy group-containing monomer (m2) within the prescribed ranges, the adhesive strength, cohesive strength and water resistance can be increased. In some embodiments, in view of water resistance, flexibility, etc., the amount of carboxy group-containing monomer (m2) used is, for instance, suitably less than 10 wt % of the entire monomer mixture, preferably 5.0 wt % or less, or may be 4.5 wt % or less, or 3.5 wt % or less.

As the N-containing monomer (m3), a monomer having a structure with no hydrogen atoms bonded to the nitrogen atom is used. By using an N-containing monomer (m3) having such a structure in combination with the carboxy group-containing monomer (m2), the adhesive strength and water resistance can be increased. The N-containing but NH-free monomer (m3) has stronger acid-base interactions with the carboxylic acids which includes acrylic acid, than an N-containing monomer with an N-bonded hydrogen atom. It is thought that these acid-base interactions increase the cohesive strength, thereby increasing the adhesive strength and water resistance. It is noted that the art disclosed herein is not limited to the above considerations. For the N-containing monomer (m3), solely one species or a combination of two or more species can be used.

While no particular limitations are imposed, a preferable N-containing monomer (m3) has a low acid dissociation constant (pKa). Presumably, the lower the pKa, the stronger the acid-base interactions with carboxylic acids, and the easier it is to obtain excellent cohesive strength. The N-containing monomer (m3) has a pKa of, for instance, 7 or lower, 5 or lower, or 3 or lower. In some preferable embodiments, in view of obtaining greater adhesive strength and water resistance, an N-containing monomer (m3) having a pKa of 1 or lower is used. The pKa is more preferably 0.5 or lower, yet more preferably 0 or lower (e.g., below 0), or may be −0.5 or lower. The minimum pKa of the N-containing monomer (m3) is −2 or higher, −1.5 or higher, or −1 or higher. By using an N-containing monomer (m3) having a pKa within these ranges, good acid-base interactions can be obtained based on the basicity. The pKa can be determined from the concentration and the hydrogen ion concentration of the substance of interest, with the hydrogen ion concentration measured using a pH meter.

As the N-containing monomer (m3), it is possible to use a monomer that has a structure with no hydrogen atoms bonded to the nitrogen atom. Examples include a monomer having an N-containing ring (an N-heterocyclic monomer), an amide group-containing monomer, and an amino group-containing monomer.

Examples of the N-heterocyclic monomer include N-vinyl-2-pyrrolidone, N-methylvinylpyrrolidone, N-vinylpyridine, N-vinylpiperidone, N-vinylpyrimidine, N-vinylpyrazine, N-vinylpyrrole, N-vinylimidazole, N-vinyloxazole, N-(meth)acryloyl-2-pyrrolidone, N-(meth)acryloyl piperidine, N-(meth)acryloylpyrrolidine, N-(meth)acryloylmorpholine, N-vinylmorpholine, N-vinyl-3-morpholinone, N-vinyl-2-caprolactam, N-vinyl-1,3-oxadin-2-one, N-vinyl-3,5-morpholindione, N-vinylisoxazole, N-vinylthiazole, N-vinylisothiazole and N-vinylpyridazine.

Examples of the amide group-containing monomer include N,N-dialkyl (meth)acrylamides such as N,N-dimethyl (meth)acrylamide, N,N-diethyl (meth)acrylamide, N,N-dipropyl (meth)acrylamide, N,N-diisopropyl (meth)acrylamide, N,N-di-n-butyl (meth)acrylamide and N,N-di-t-butyl (meth)acrylamide.

Examples of the amino group-containing monomer include N,N-dialkylaminoalkyl (meth)acrylates such as N,N-dimethylaminoethyl (meth)acrylate.

In some preferable embodiments, as the N-containing monomer (m3), an N-containing cyclic monomer having a cyclic nitrogen atom (an N-heterocyclic monomer) is used. The use of the N-heterocyclic monomer can bring about greater adhesive strength and water resistance. This can be because the structure with an N-containing ring provides a distance that facilitates the acid-base interactions to increase the cohesive strength more effectively. Favorable examples of the N-heterocyclic monomer include N-vinyl-2-pyrrolidone (NVP) and N-(meth)acryloylmorpholine. In particular, NVP is highly biocompatible and is suitable for a dermal PSA. For the N-heterocyclic monomer, solely one species or a combination of two or more species can be used.

In the embodiment using an N-heterocyclic monomer as the N-containing monomer (m3), the amount of N-heterocyclic monomer used in the entire N-containing monomer (m3) is not particularly limited. In some embodiments, in view of effectively obtaining the effect of using the N-heterocyclic monomer, the amount of N-heterocyclic monomer in the entire N-containing monomer (m3) may be, for instance, 10 wt % or greater, suitably 50 wt % or greater, is preferably 70 wt % or greater, or 90 wt % or greater (e.g., 95 wt % to 100 wt %).

The amount of N-containing monomer (m3) (i.e., the copolymerization ratio of N-containing monomer (m3) in acrylic polymer) is not particularly limited. It can be, for instance, 0.1 wt % or more of the entire monomer mixture, preferably 0.3 wt % or more, more preferably 0.5 wt % or more, yet more preferably 0.8 wt % or more, particularly preferably 1.0 wt % or more, or may be 1.5 wt % or more (e.g., 1.8 wt % or more). By increasing the amount of N-containing monomer (m3) within the prescribed ranges, the adhesive strength, cohesive strength and water resistance can be increased. In some embodiments, in view of water resistance, flexibility, etc., the amount of N-containing monomer (m3) used is, for instance, suitably less than 10 wt % of the entire monomer mixture, preferably 5.0 wt % or less, more preferably 3.0 wt % or less, yet more preferably 2.5 wt % or less, or may be 2.0 wt % or less, 1.5 wt % or less, or 1.2 wt % or less.

The ratio of carboxy group-containing monomer (m2) to N-containing monomer (m3) is not particularly limited and is suitably set to obtain desirable adhesive strength and water resistance. In some embodiments, the weight ratio (m2/m3) of carboxy group-containing monomer (m2) to N-containing monomer (m3) may be, for instance, 0.1 or higher, is suitably 0.5 or higher, preferably 1 or higher (e.g., above 1), more preferably 1.5 or higher, yet more preferably 2.0 or higher, or may be 2.5 or higher, 3.0 or higher, or 3.5 or higher. In some embodiments, the ratio (m2/m3) may be, for instance, 10 or lower, is suitably 7 or lower, preferably 5 or lower, more preferably 4 or lower, or may be 3 or lower, or 2.5 or lower.

In some embodiments, the monomers forming the acrylic polymer further include a silane-based monomer. The acrylic polymer including a silane-based monomer as a monomer unit can introduce crosslinking structures among polymer particles through silanol condensation (condensation reactions of silanol groups) in the water-dispersed PSA, and the crosslinks among polymer particles can bring about increases in cohesive strength, adhesive strength and water resistance. Silane-based monomers are also referred to as silane coupling agents.

As the silane-based monomer, an alkoxysilyl group-containing monomer is preferably used. An alkoxysilyl group-containing monomer is typically an ethylenically unsaturated monomer having at least one (preferably two or more, e.g., two or three) alkoxysilyl group(s) per molecule. Examples of the silane-based monomer include 3-(meth)acryloxypropyltrimethoxysilane, 3-(meth)acryloxypropyltriethoxysilane, 3-(meth)acryloxypropylmethyldimethoxysilane and 3-(meth)acryloxypropylmethyldiethoxysilane. For the silane-based monomer, solely one species or a combination of two or more species can be used.

In the embodiment where the monomer mixture includes a silane-based monomer, the amount of alkoxysilyl group-containing monomer in the entire monomer mixture is, for instance, suitably 0.001 wt % or greater, or preferably 0.01 wt % or greater. In some embodiments, in view of enhancing the adherend adhesion, etc., the amount of silane-based monomer is, for instance, suitably 0.5 wt % or less, preferably 0.2 wt % or less, or may be 0.1 wt % or less, or 0.05 wt % or less.

The monomers forming the acrylic polymer may include, as necessary, other monomer(s) that are copolymerizable with the alkyl (meth)acrylate (other copolymerizable monomer(s)) and are different from the carboxy group-containing monomer (m2), N-containing monomer (m3) and silane-based monomer. As the other copolymerizable monomer(s), it is possible to suitably use a monomer having a functional group such as a hydroxy group, or a monomer having a relatively high homopolymer glass transition temperature (e.g., 10° C. or higher). For the other copolymerizable monomer(s), solely one species or a combination of two or more species can be used.

Non-limiting examples of the other copolymerizable monomers include acid anhydride group-containing monomers, hydroxy group-containing monomers (e.g., 2-hydroxyethyl acrylate and 4-butoxybutyl acrylate), sulfonate or phosphate group-containing monomers, epoxy group-containing monomers, cyano group-containing monomers, isocyanate group-containing monomers, alkoxy group-containing monomers, vinyl esters, vinyl ethers, aromatic vinyl compounds, olefins, (meth)acrylates having alicyclic hydrocarbon groups, (meth)acrylates having aromatic hydrocarbon groups; as well as heteroring-containing (meth)acrylates such as tetrahydrofurfuryl (meth)acrylate, halogen atom-containing (meth)acrylates such as vinyl chloride and fluorine atom-containing (meth)acrylates, silicon atom-containing (meth)acrylates such as silicone (meth)acrylate, (meth)acrylic esters obtained from terpene compound derivative alcohols, and polyfunctional monomers (monomers having at least two polymerizable functional groups with unsaturated double bonds such as (meth)acryloyl groups and vinyl groups). Other possible examples of the other copolymerizable monomer(s) include carboxy group-containing monomers different from acrylic acid (m2a) and a monomer (m2b), and N-containing monomers with N-bonded hydrogen atoms (e.g., amide group-containing monomers and amino group-containing monomers having a structure with an N-bonded hydrogen).

The other copolymerizable monomer(s) can be used in suitable amounts as long as the effects of the invention are not impaired. When the monomer mixture of the acrylic polymer includes the other copolymerizable monomer(s), the amount of other copolymerizable monomer(s) in the entire monomer mixture is suitably about 30 wt % or less, or may be 10 wt % or less, 3 wt % or less, or less than 1 wt % (e.g., less than 0.1 wt %). The monomer mixture may be essentially free of other copolymerizable monomers. The PSA disclosed herein can realize high cohesive strength because of the combined use of the carboxy group-containing monomer (m2) and N-containing monomer (m3). Thus, there is no need to rely on hydroxy group-containing monomers used for crosslinking reactions or cohesive strength enhancement. Accordingly, the art disclosed herein can be preferably implemented in an embodiment where the amount of hydroxy group-containing monomer in the monomer mixture for the acrylic polymer is less than 3 wt % (e.g., less than 1 wt %, or even less than 0.1 wt %), or in an embodiment where the monomer mixture is essentially free of hydroxy group-containing monomers.

The acrylic polymer can be synthesized by emulsion polymerization. The emulsion polymerization method is not particularly limited. It can be carried out by suitably adopting various monomer supply methods, polymerization conditions, materials used, etc., similar to those of conventional emulsion polymerization. For instance, as the monomer supply method, a suitable method can be employed among the all-at-once method where all the starting monomer material (monomer mixture) is supplied in one portion, gradual (dropwise) supply method, portion-wise (dropwise) supply method, etc. It is preferable to supply the starting monomer material as a dispersion (emulsion) of a portion or all thereof emulsified in water. For instance, for dropwise supply, the starting monomer material is preferably added dropwise in the form of emulsion. The polymerization temperature can be, for instance, about 30° C. or higher (typically 50° C. or higher), or suitably about 100° C. or lower (typically 80° C. or lower).

The initiator used in polymerization can be suitably selected among heretofore known polymerization initiators according to the polymerization method. Possible examples include azo-based polymerization initiator, peroxide-based polymerization initiator, a redox-based polymerization initiator by combination of a peroxide and a reducing agent, and substituted ethane-based polymerization initiator. In emulsion polymerization, a water-soluble polymerization initiator is preferably used. For the polymerization initiator, solely one species or a combination of two or more species can be used. The amount of polymerization initiator used can be selected, for instance, in the range of about 0.005 part to 1 part (typically about 0.01 part to 1 part) by weight per 100 parts by weight of monomers. The emulsifier used in emulsion polymerization is not particularly limited. Known anionic surfactants, nonionic surfactants and the like can be used. In some embodiments, in view of increasing water resistance, it is preferable to use a surfactant (reactive surfactant) having a radically-polymerizable functional group. For the emulsifier, solely one species or a combination of two or more species can be used.

Possible examples of the reactive emulsifier include anionic and nonionic surfactants having structures with radically-polymerizable functional groups introduced therein. In view of ease of emulsification such as polymerization stability during emulsion polymerization, etc., it is preferable to use an anionic reactive emulsifier. Examples of the radically-polymerizable functional group include vinyl group, propenyl group, isopropenyl group, vinyl ether group (vinyloxy group), and allyl ether group (allyloxy group). The concept of propenyl group here encompasses 1-propenyl group (CH₃—CH═CH—) and 2-propenyl group (CH₂═CH—CH₂-which may also be called allyl group).

Examples of anionic reactive emulsifiers include polyoxyethylene (allyloxymethyl) alkyl ether sulfates (e.g., ammonium salts), polyoxyethylene nonyl propenyl phenyl ether sulfate (e.g., ammonium salt), alkyl allyl sulfosuccinates (e.g., sodium salts), methacryloxy polyoxypropylene sulfuric acid ester salts (e.g., sodium salts), and polyoxyalkylene alkenyl ether sulfates (e.g., ammonium salt in which the alkenyl group terminal is an isopropenyl group). When the anionic reactive emulsifier is in a salt form, the salt can be, for instance, a metal salt (e.g., sodium salt) or a non-metal salt (e.g., ammonium salt, amine salt, etc.).

Examples of nonionic reactive emulsifiers include polyoxyethylene nonyl propenyl phenyl ether.

When the starting monomer mixture is emulsion-polymerized in the presence of a reactive emulsifier having a radically-polymerizable functional group, the reactive emulsifier can react and be incorporated in the acrylic polymer. Because the reactive emulsifier incorporated in the acrylic polymer is restricted in its movement within a PSA layer, it is less likely to migrate to the PSA layer surface. Thus, the use of a reactive emulsifier can inhibit the migration of the emulsifier to the PSA layer surface. This is advantageous in inhibiting a decrease in water-resistant adhesive strength in case of water occurrence on the PSA layer surface, and is especially preferable as it can increase sweat resistance.

In emulsion polymerization, the amount of emulsifier used per 100 parts by weight of monomers can be, for instance, 0.2 part by weight or greater, 0.5 part by weight or greater, or 1.0 part by weight or greater, or 1.5 parts by weight or greater. In view of enhancing the water resistance, in some embodiments, the amount of emulsifier used per 100 parts by weight of monomers is suitably less than 10 parts by weight, preferably 5 parts by weight or less, more preferably 3 parts by weight or less, or yet more preferably 2.5 parts by weight or less.

In the polymerization, various kinds of heretofore known chain transfer agent (which may also be thought as molecular weight-adjusting agent or polymerization degree-adjusting agent) can be used as necessary. As the chain transfer agent, mercaptans can be preferably used, such as n-dodecyl mercaptan, t-dodecyl mercaptan, thioglycolic acid and α-thioglycerol. Alternatively, a chain transfer agent free of sulfur atoms (a sulfur-free chain transfer agent) can be used as well. For the chain transfer agent, solely one species or a combination of two or more species can be used. When using a chain transfer agent, the amount used per 100 parts by weight of monomers can be, for instance, about 0.01 part to 1 part by weight.

The emulsion polymerization enables preparation of an acrylic polymer emulsion (a polymer syrup in an emulsion form in which the acrylic polymer is dispersed in water). The water-dispersed PSA composition disclosed herein can be preferably produced using the polymer syrup as is or after appropriate work-up.

### (Crosslinking Agent)

The PSA composition disclosed herein may include a crosslinking agent as necessary. The species of crosslinking agent is not particularly limited and can be suitably selected and used among heretofore known crosslinking agents. Examples of such crosslinking agents include isocyanate crosslinking agent, epoxy crosslinking agent, oxazoline crosslinking agent, aziridine crosslinking agent, melamine crosslinking agent, peroxide crosslinking agent, and metal chelate crosslinking agent. For the crosslinking agent, solely one species or a combination of two or more species can be used.

The amount of crosslinking agent is not particularly limited. For instance, it can be selected in the range of about 10 parts by weight or less (e.g., about 0.001 part to 10 parts by weight, preferably about 0.01 part to 5 parts by weight) per 100 parts by weight of acrylic polymer. In some embodiments, the amount of crosslinking agent used per 100 parts by weight of acrylic polymer is less than 1 part by weight, or may be less than 0.5 part by weight, or less than 0.1 part by weight (e.g., 0.05 part by weight). In some embodiments, the PSA composition may be essentially free of a crosslinking agent. According to the art disclosed herein, the monomer composition of the acrylic polymer can be designed to obtain high cohesive strength by using a small amount of crosslinking agent or by using essentially no crosslinking agent.

### (Other Optional Components)

The PSA composition disclosed herein can optionally include a tackifier resin for increasing adhesive strength or like purpose. As the tackifier resin, it is possible to use one, two or more species selected among tackifier resins such as phenolic tackifier resins, terpene-based tackifier resins, modified terpene-based tackifier resins, rosin-based tackifier resins, hydrocarbon-based tackifier resins, epoxy-based tackifier resins, polyamide-based tackifier resins, elastomer-based tackifier resins, and ketone-based tackifier resins. When the PSA composition comprises a tackifier resin, the amount of the tackifier resin used per 100 parts by weight of acrylic polymer is, for instance, in the range of about 1 part to 30 parts by weight, or may be 10 parts by weight or less. In some embodiments, the PSA composition is essentially free of a tackifier resin. According to the art disclosed herein, the PSA composition (and thus the PSA) can realize high adhesive strength without using a tackifier resin.

The PSA composition may include, as necessary, various additives generally used in the field of PSA compositions such as a leveling agent, cross-linking auxiliary agent, plasticizer, softening agent, filler, coloring agent (dye, pigment), antistatic agent, anti-aging agent, UV absorber, antioxidant, photostabilizer, and dispersant. In an embodiment where the PSA disclosed herein is applied to human skin, the PSA may include a component that reduces skin irritation, such as a carboxylic acid ester and an aliphatic acid ester. As for the various additives, heretofore known species can be used by typical methods. As they do not particularly characterize the present invention, their details are omitted.

In some embodiments, the amounts of components other than acrylic polymer (or “non-acrylic components”) may be limited. With a PSA having a low amount of non-acrylic components, it is possible to prevent or reduce their effects on adherends such as the skin. For instance, when the adherend is the skin, the inclusion of skin-irritating components can be reduced. From such a viewpoint, while no particular limitations are imposed, the amount of non-acrylic components (non-volatiles, in particular) in the PSA composition (and thus the PSA) is, for instance, suitably about 10 wt % or less, preferably about 5 wt % or less, more preferably about 3 wt % or less, or may be less than 1.0 wt %. The art disclosed herein can be preferably implemented in a composition having a limited amount of non-acrylic components.

### <PSA>

The PSA disclosed herein can be formed, for instance, using an aforementioned PSA composition, by a heretofore known method. For instance, the PSA composition can be applied to a releasable surface (release face) and allowed to dry to form a layer of PSA (a PSA layer). In a PSA sheet having a support substrate, for instance, a direct method can be employed, by which the PSA composition is directly provided (typically applied) to the support substrate and allowed to dry to form a PSA layer. A transfer method can also be employed, by which the PSA composition is provided to a releasable surface (release face) and allowed to dry to form a PSA layer on the surface, and the PSA layer is transferred onto a support substrate. As the release face, for instance, a release liner surface can be preferably used. The PSA layer disclosed herein is typically formed in a continuous form, but it is not limited to such a form. For instance, the PSA layer may be formed in a regular or random pattern of dots, stripes, etc.

The PSA composition can be applied by using a heretofore known coater such as a gravure roll coater, die coater and bar coater. Alternatively, the PSA composition can be applied by impregnation, curtain coating, etc. From the standpoint of accelerating the crosslinking reaction, improving production efficiency, and so on, it is preferable to dry the PSA composition by heating. The drying temperature may be, for instance, about 40° C. to 150° C., or is preferably about 60° C. to 130° C. After dried, the PSA composition can be further aged for adjustment of component migration in the PSA, progress of crosslinking reaction, relaxation of possible strain in the PSA, etc.

### (25° C. Storage Modulus)

While no particular limitations are imposed, in some embodiments, the PSA (layer) may have a storage modulus at 25° C. (25° C. storage modulus) of, for instance, less than 5 MPa, or less than 3 MPa. In some preferable embodiments, the PSA (layer) has a 25° C. storage modulus of less than 1.00 MPa, more preferably less than 0.80 MPa, yet more preferably 0.70 MPa or less, or may be 0.60 MPa or less (e.g., 0.50 MPa or less). The PSA with such 25° C. storage modulus has excellent flexibility. The art disclosed herein can combine high adhesive strength and water resistance while having such great flexibility. Thus, for instance, when used on the skin, the flexibility can bring about highly tight interfacial contact to provide tighter adhesion to the skin. In some embodiments, in view of good adhesive properties (adhesive strength, holding power, etc.), the 25° C. storage modulus is, for instance, 0.03 MPa or greater, or may be 0.10 MPa or greater, or 0.30 MPa or greater.

### (37° C. Storage Modulus)

While no particular limitations are imposed, in some embodiments, the PSA (layer) may have a storage modulus at 37° C. (37° C. storage modulus) of, for instance, less than 3 MPa, or less than 1 MPa. In some preferable embodiments, the PSA (layer) has a 37° C. storage modulus of less than 0.80 MPa, more preferably less than 0.50 MPa, or may be 0.40 MPa or less. For instance, when used on the skin, the PSA with such 37° C. storage modulus shows good flexibility and can adhere more tightly to the adherend skin. In some embodiments, in view of good adhesive properties (adhesive strength, holding power, etc.), the 37° C. storage modulus is, for instance, 0.01 MPa or greater, or may be 0.10 MPa or greater, or 0.20 MPa or greater.

### (Tan & Peak-Top Temperature)

The tan δ peak-top temperature of the PSA (layer) is set to produce target adhesive properties and is not limited to a specific range. The tan δ peak-top temperature of a PSA (layer) refers to the loss tangent tan δ (G″/G′) peak temperature (° C.) determined from the storage modulus G′ and loss modulus G″ by dynamic viscoelastic analysis. In some embodiments, the PSA (layer) can have a tan δ peak-top temperature of, for instance, 0° C. or lower, or −20° C. or lower. In some preferable embodiments, the tan δ peak-top temperature is suitably below −30° C., more preferably below −35° C. (e.g., −36° C. or lower), or may be −40° C. or lower. The PSA showing such a tan δ peak-top temperature is flexible enough to readily conform to the adherend surface. For instance, when used on the skin, it exhibits good flexibility and can adhere more tightly to the adherend skin. In some embodiments, in view of cohesive strength, etc., the tan δ peak-top temperature is suitably −50° C. or higher, or may be −45° C. or higher.

The viscoelastic properties (25° C. and 37° C. storage moduli, tan δ peak-top temperature) of the PSA (layer) can be determined by dynamic viscoelastic analysis. In particular, they are determined by the methods described later in Examples. Based on the content of this description, the viscoelastic properties can be adjusted through the monomer composition and polymerization conditions of the acrylic polymer, PSA components and composition, etc.

### <PSA Sheet>

### (Structure)

The PSA sheet disclosed herein is not particularly limited as long as it has a PSA layer formed from an aforementioned PSA composition. It can be a PSA sheet with substrate having the PSA layer on one or each face of a non-releasable substrate (support substrate), or a PSA sheet without substrate in which the PSA layer is supported on a release liner (i.e., a PSA sheet free of a non-releasable substrate, typically a PSA sheet formed of a PSA layer). The concept of PSA sheet herein may encompass so-called PSA sheet, PSA tape, PSA labels, PSA film, etc. The PSA sheet disclosed herein can be in a roll or in a flat sheet. Alternatively, the PSA sheet may be processed into various shapes.

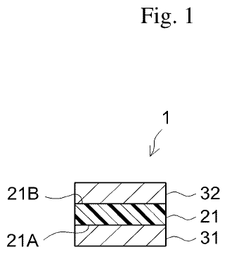

FIG. 1 shows a structural example of the PSA sheet (double-faced PSA sheet without substrate). PSA sheet 1 shown in FIG. 1 has a structure in which both faces (first and second adhesive surfaces 21A and 21B) of a PSA layer 21 without substrate are protected, respectively, with release liners 31 and 32 with at least the PSA layer side of which being a release face. Alternatively, it may have a structure in which release liner 32 is omitted, and PSA sheet 1 is layered with release liner 31 having release faces on both sides so that, when wound in a roll, the second adhesive face 21B makes contact and is protected with the backside of release liner 31.

### (Psa Layer)

The PSA layer thickness is not particularly limited. The PSA layer thickness is typically about 300 μm or less, suitably about 100 μm or less, preferably about 80 μm or less, or may be 60 μm or less. With such a limited thickness, the PSA layer disclosed herein can exhibit high adhesive strength as aimed. A PSA layer with a limited thickness can well adopt to demands for thinning and lightening. The minimum PSA layer thickness is not particularly limited. In view of adhesive strength and adherend conformability, it is, for instance, about 1 μm or greater, suitably about 10 μm or greater, preferably 20 μm or greater, or may be, for instance, 30 μm or greater. In an embodiment using a highly flexible PSA layer, the PSA layer having a thickness in these ranges is likely to obtain conformability to the adherend (e.g., human skin). When the PSA sheet is formed of a PSA layer without a substrate (i.e., a PSA sheet without substrate), the thickness ranges correspond to the PSA sheet thickness.

### (Substrate)

In some embodiments, the PSA sheet may be in the form of a PSA sheet with substrate, having a PSA layer on one or each face of a support substrate (or simply “substrate” hereinafter). Possible examples of the substrate supporting (backing) the PSA layer include films of polyolefins (polyethylene, polypropylene, ethylene-propylene copolymer, etc.), films of polyesters (polyethylene terephthalate (PET), etc.), vinyl chloride resin film, vinyl acetate resin film, polyimide resin film, polyamide resin film, fluororesin film, and other plastic films such as cellophane; paper materials such as Japanese paper, kraft paper, glassine paper, high-grade paper, synthetic paper, and top-coated paper; fabrics such as woven and nonwoven fabrics made from various fibrous materials, either alone or in combination; rubber sheets made from natural rubber, butyl rubber, etc.; foam sheets made from foamed polyurethane, foamed polychloroprene rubber, etc.; metal foils such as aluminum foil and copper foil; and composites of these. The plastic films may be non-oriented or oriented (mono-oriented or biaxially oriented). The substrate may have a single layer form or a laminated form.

In some embodiments, as the substrate, nonwoven and woven fabrics, other porous films and the like are used. When using nonwoven and woven fabrics, their materials are not particularly limited. It is possible to use materials formed from one, two or more species among natural fibers such as cotton, hemp, and wool; cellulose-based fibers such as rayon and acetate; polyamide fibers such as vinylon and nylon; polyolefin fibers such as polyethylene and polypropylene; polyester fibers such as polyethylene terephthalate (PET); synthetic fibers such as polyurethane fibers; and the like. Porous films include porous resin films formed of polypropylene, polyethylene, PET, polyurethane, etc. Porous films include porous resin films formed of polypropylene, polyethylene, PET and polyurethane. In particular, nonwoven fabrics are preferable and nonwoven fabrics formed from synthetic fabrics such as polyester fabric are more preferable. These substrate materials have suitable breathability and moisture permeability, and can also have suitable elasticity/non-elasticity. Thus, for instance, they can be preferably used for applications to human skin.

As necessary, the substrate may include various additives such as a filler (inorganic filler, organic filler, etc.), anti-aging agent, antioxidant, UV absorber, anti-static agent, slip agent, plasticizer, and colorant (pigment, dye, etc.). The substrate surface (especially, the surface on which the PSA layer is formed) may be subjected to a known or conventional surface treatment such as corona discharge treatment, plasma treatment, and primer application. Such a surface treatment may enhance the substrate-anchoring properties of the PSA layer, for instance.

The substrate thickness can be suitably selected in accordance with the purpose. It is typically about 10 μm or greater, or may be, for instance, 20 μm or greater, or 30 μm or greater. With increasing substrate thickness, the strength of the substrate and PSA sheet tend to increase, making them easier to use and handle. The maximum substrate thickness is not particularly limited. In some embodiments, for instance, it is suitably about 1 mm or less, or may be about 500 μm or less, about 300 μm or less, or 100 μm or less. Limiting the substrate thickness tends to increase the conformability to the adherend surface structure (unevenness, etc.).

### (Release Liner)

The release liner is not particularly limited. Possible examples include a release liner in which a surface of a liner substrate such as resin film or paper is release-treated, and a release liner formed from a low-adhesive material such as fluoropolymer (polytetrafluoroethylene, etc.) and polyolefinic resin (polyethylene, polypropylene, etc.). For the release treatment, for instance, a release agent such as silicone-based and long-chain alkyl-based release agents can be used. In some embodiments, release-treated resin film can be preferably used as the release liner.

### (Adhesive Strength)

While no particular limitations are imposed, in some embodiments, the PSA sheet has an adhesive strength of preferably about 3.0 N/10 mm or greater, more preferably about 3.5 N/10 mm or greater, or yet more preferably about 4.0 N/10 mm or greater. The PSA sheet having such high adhesive strength adheres well to an adherend and is less likely to shift or peel off while on the adherend. The maximum adhesive strength is not particularly limited. In some embodiments, it can be less than 10 N/10 mm, or 6 N/10 mm or less. The PSA sheet with adhesive strength limited up to such a prescribed value can reduce the peel load on and damage to an adherend when peeled off from the adherend. The adhesive strength is the 180° peel strength determined using an acrylic plate or Bakelite plate as the adherend. The adhesive strength on acrylic plate (on-acrylic-plate adhesive strength) and adhesive strength on Bakelite plate (on-Bakelite-plate adhesive strength) can be measured, specifically by the method described later in the working examples. The PSA sheet disclosed herein preferably has an aforementioned adhesive strength value for at least one (preferably both) of the on-acrylic-plate adhesive strength and the on-Bakelite-plate adhesive strength. The PSA sheet having on-acrylic-plate and on-Bakelite-plate adhesive strength values in the aforementioned ranges can tightly adhere to the skin. In some embodiments, one of the on-acrylic-plate or on-Bakelite-plate adhesive strength can be about 4.5 N/10 mm or greater, or about 5.0 N/10 mm or greater.

While no particular limitations are imposed, in some embodiments, the PSA sheet has a water-resistant adhesive strength of preferably about 1.0 N/10 mm or greater, more preferably about 1.5 N/10 mm or greater, or yet more preferably about 2.0 N/10 mm or greater. The PSA sheet exhibiting such water-resistant adhesive strength has good water resistance and can thus be used preferably as a highly reliable adhesive means in various applications which may involve contact with water. It is especially suited for dermal use requiring sweat resistance. The maximum water-resistant adhesive strength is not particularly limited. In some embodiments, it can be less than 5 N/10 mm. The water-resistant adhesive strength is the 180° peel strength determined using an acrylic plate as the adherend after the adhesive face of the PSA sheet is immersed in water for one hour. It can be measured specifically by the method described later in the working examples.

### (Total Thickness)

The total thickness of the PSA sheet (including the PSA layer and substrate, but not a release liner if any) disclosed herein is not particularly limited. In some preferable embodiments, the total PSA sheet thickness can be, for instance, about 1 mm or less. In view of handling properties, etc., it can be about 500 μm or less, about 300 μm or less, or about 100 μm or less. PSA sheets with limited thickness are suitable for applications desiring light weight and small thickness. The minimum PSA sheet thickness is not particularly limited. In view of adhesive properties (adhesive strength, etc.), handling properties, etc., in some embodiments, it is, for instance, suitably about 10 μm or greater, preferably about 20 μm or greater, more preferably about 30 μm or greater, or may be about 50 μm or greater.

### <Applications>

The purposes of the PSA composition, PSA and PSA sheet disclosed here are not particularly limited, and it can be used in applications. The PSA and PSA sheet disclosed herein have high adhesive strength and good water resistance, and can thus be used preferably as highly reliable adhesive means in various applications which may involve contact with water. The PSA can have high adhesive strength and good water resistance along with flexibility. Based on the flexibility, adhesive strength and water resistance, it can be preferably used as a dermal PSA or dermal PSA sheet that adheres well to the skin and has good sweat resistance. Such PSAs and dermal PSA sheets can be preferably used as PSAs and PSA sheets for medical, healthcare and sports applications, such as bandages, surgical tapes and kinesiology tapes.

### EXAMPLES

Several working examples related to the present invention are described below, but these specific examples are not to limit the present invention. In the description below, “parts” and “%” are by weight unless otherwise specified.

### Example 1

To a flask equipped with a reflux condenser, thermometer and dropping funnel, were added 85 g of water and 10 g of reactive emulsifier A (product name AQUALON KH-1025 available from Dai-ichi Kogyo Seiyaku Co., Ltd.; 25% NV). Separately, were added the following together and emulsified in a high-pressure homogenizer to obtain an emulsion: as monomers, 350 g of 2-ethylhexyl acrylate (2EHA), 7.3 g of acrylic acid (AA), 3.7 g of N-vinylpyrrolidone (NVP) and 3.7 g methacrylic acid (MAA); 0.18 g of a silane-based monomer (product name KBM-503 available from Shin-Etsu Chemical Co., Ltd.; 3-methacryloxypropyltrimethoxysilane), 0.18 g of t-lauryl mercaptan as a chain transfer agent, 25 g of reactive emulsifier A (product name AQUALON KH-1025), and 140 g of water. Into the flask, 40 g of the emulsion was added and the flask was purged with nitrogen for one hour. The mixture was then heated to 60° C. To this, was added 0.1 g of a water-soluble polymerization initiator (product name VA-057 available from FUJIFILM Wako Pure Chemical Corporation) to initiate polymerization. After one hour, was added 0.1 g of the water-soluble polymerization initiator (product name VA-057) and was added dropwise a prescribed amount of the emulsion obtained above over 3 hours to carry out emulsion polymerization. The reaction mixture was then heated to 70° C. and further maintained at the temperature for 3 hours. After cooled to room temperature, to 100 g of the aqueous dispersion after the polymerization reaction, while stirring, was added 1.0 g of 10% ammonia water. After thorough mixing, the mixture was defoamed using product name AWATORI RENTARO available from THINKY Corporation to prepare a water-dispersed PSA composition according to this example.

To the release-treated surface of a 50 μm thick release liner (product name PET50-SCA1 available from Fujiko Co., Ltd.; PET film release-treated with silicone on one side), was applied the water-dispersed PSA composition and allowed to dry at 130° C. for 3 minutes to form a 50 μm thick PSA layer. The exposed adhesive face of the PSA layer was covered and protected with a 38 μm thick release liner (product name MRE38 available from Mitsubishi Chemical Corporation; PET film release-treated with silicone on one side). In this manner, was obtained a PSA sheet formed of a PSA layer with each side protected with a release liner.

Table 1 shows the relative amounts of the components used in the water-dispersed PSA composition according to this example based on their parts. Table 1 also shows the acid dissociation constant (pKa) and water solubility at 20° C. of each component.

### Examples 2 to 5 and Comparative Examples 1, 3 to 5

The components used were changed as shown in Table 1. Otherwise in the same manner as Example 1, was prepared a water-dispersed PSA composition according to each example. Using the resulting water-dispersed PSA composition, was obtained a PSA sheet according to each example.

In Table 1, HOA-MS represents 2-acryloyloxyethyl succinic acid; ACMO, N-acryloylmorpholine; DEAA, diethylacrylamide; DMAPAA, dimethylaminopropylacrylamide; and NIPAM, N-isopropylacrylamide.

### Comparative Example 2

To a monomer mixture of 335 g of 2EHA, 18 g of methyl methacrylate (MMA), 7 g of AA, 3.6 g of MAA and 0.04 g of t-lauryl mercaptan, were added 16 g of emulsifier B (product name LATEMUL PD-420 available from Kao Corporation) and 170 g of water. The resulting mixture was emulsified, using an emulsifying machine. Using the same polymerization apparatus as in Example 1, to the flask, were added 25 g of water and 0.3 g of ammonium persulfate as a polymerization initiator. After nitrogen purging, the temperature was raised to 55° C. After the temperature elevation, the emulsion was added gradually from a dropping funnel over 3 hours to carry out dropwise polymerization. After maintaining the same temperature for 2 more hours, the temperature was raised to 70° C. and further maintained for 3 hours. After cooling to room temperature, the pH was adjusted to 8.0 using 10% ammonia water, and the aggregates were filtered using a Teflon® mesh to obtain a water-dispersed PSA composition according to this example. Using this water-dispersed PSA composition, but otherwise in the same manner as Example 1, was obtained a PSA sheet formed of a 50 μm thick PSA layer.

### <Evaluations>

### [180° Peel Strength (Adhesive Strength)]

With respect to the PSA sheet according to each example, the release liner covering one (first) adhesive face was removed and a 25 μm thick PET film (product name LUMIRROR S10 available from Toray Industries, Inc.) was adhered as backing to obtain a measurement sample. The measurement sample was cut to a 5 cm×1 cm size. In an environment at room temperature (23° C.), the other (second) adhesive face was exposed, adhered to an acrylic plate as the adherend, and press-bonded with a 2 kg roller moved back and forth once. In the same environment, using a tensile tester (product name AUTOGRAPH AG-Xplus, HS 6000 mm/min high-speed model (AG-50NX plus) available from Shimadzu Corporation), while peeling off the PSA sheet from the adherend at a peel angle of 180° at a tensile speed of 300 mm/min, the load was measured to determine the peel strength (N/10 mm) on acrylic plate (on-acrylic-plate adhesive strength). As the acrylic plate, was used product name ACRYLITE available from Mitsubishi Chemical Corporation.

Using a Bakelite plate as the adherend, but otherwise in the same manner as the above, the peel strength was measured to determine the peel strength (N/10 mm) on Bakelite plate (on-Bakelite-plate adhesive strength). As the Bakelite plate, was used a phenol laminate (phenolic paper) available from Standard TestPiece Co., Ltd.

### [Water-Resistant Adhesive Strength]

With respect to the PSA sheet according to each example, the release liner covering one (first) adhesive face was removed and a 25 μm thick PET film (product name LUMIRROR S10 available from Toray Industries, Inc.) was adhered as backing to obtain a measurement sample. The measurement sample was cut to a 5 cm×1 cm size. The other (second) adhesive face was exposed and the exposed adhesive face was immersed in water for 1 hour. The measurement sample was removed from the water, adhered to an acrylic plate as the adherend in an environment at room temperature, and press-bonded with a 2 kg roller moved back and forth once. Excess water residue was removed. Subsequently, in the same environment, using the tensile tester, while peeling off the PSA sheet from the adherend at a peel angle of 180° at a tensile speed of 300 mm/min, the load was measured to determine the peel strength (N/10 mm) on acrylic plate (on-acrylic-plate water-resistant adhesive strength).

### [Adhesive Strength on Skin]

The PSA sheet according to each example was cut to 5 cm×5 cm. The release liner covering one (first) adhesive face was then removed. The exposed adhesive face was adhered to a 5 cm×25 cm cut piece of polyester spunlace nonwoven fabric (product name SONTARA #8010 available from Nissei Co., Ltd.; 44 g/m² weight) to obtain a measurement sample. From the measurement sample, was removed the release liner covering the other adhesive face. In an environment at room temperature, the exposed adhesive face of the measurement sample was adhered to the inner arm surface of a test subject and lightly pressed with the opposite hand for 10 seconds. Subsequently, in the same environment, using the tensile tester, while peeling off the PSA sheet from the arm at a peel angle of 180° at a tensile speed of 300 mm/min, the load was measured to determine the peel strength (N/50 mm) (on-skin adhesive strength).

### [Sweat-Resistant Adhesive Strength]

By the same method as for the on-skin adhesive strength, using a PSA sheet and a polyester spunlace nonwoven fabric, was obtained a measurement sample. The entire inner arm of the test subject was lightly wiped with a wiping tissue wet with artificial sweat (available from Hayashi Pure Chemical Ind., Ltd.; basic, pH=8.0 (25° C.)) to deposit droplets of the artificial sweat until visible on the inner arm surface. The release liner covering the other adhesive face was then peeled off from the measurement sample. In an environment at room temperature, the exposed adhesive face of the measurement sample was adhered to the inner arm (where wet with the artificial sweat) of the test subject and lightly pressed with the opposite hand for 10 seconds. Subsequently, in the same environment, using the tensile tester, while peeling off the PSA sheet from the arm at a peel angle of 180° at a tensile speed of 300 mm/min, the load was measured to determine the peel strength (N/50 mm) (sweat-resistant on-skin adhesive strength).

### [Method for Viscoelastic Analysis]

PSA layers were laminated to a thickness of 500 μm. This was autoclaved (50° C., 0.5 atm, 15 minutes) and punched out to q 5 mm to prepare a measurement sample. The measurement sample was set in a dynamic viscoelastic analyzer (ARES-G2 available from TA Instruments, Inc.) and measurements were made under the following conditions to determine the storage moduli G′ (MPa) at 25° C. and 37° C. as well as the peak-top temperature of loss tangent tan δ (G″/G′).

### (Conditions)

Measurement mode: shear mode
Temperature range: −50° C. to 150° C.
Heating rate: 5° C./min
Frequency: 1 Hz

Table 1 outlines each example and Table 2 shows the evaluation results. In Table 1, “-” means not measured or unknown.

TABLE 1

| | | | | Composition (parts) | | | | | | | | | |
|---|---|---|---|---|---|---|---|---|---|---|---|---|---|
| | | | Water solubility | | | | | | Comp. | Comp. | Comp. | Comp. | Comp. |
| | | pKa | (g/100 mL) | Ex. 1 | Ex. 2 | Ex. 3 | Ex. 4 | Ex. 5 | Ex. 1 | Ex. 2 | Ex. 3 | Ex. 4 | Ex. 5 |
| Monomers | 2EHA | — | <0.1 | 96 | 94 | 95 | 96 | 96 | 95 | 92 | 96 | 96 | 96 |
| | AA | 4.25 | Miscible | 2 | 2 | 2 | 2 | 2 | 5 | 2 | 3 | 2 | 2 |
| | MAA | 4.66 | 9.7 | 1 | 2 | | 1 | 1 | | 1 | | 1 | 1 |
| | MMA | — | 1.6 | | | | | | | 5 | | | |
| | HOA-MS | 4.35 | 3.9 | | | 2 | | | | | | | |
| | NVP | −0.34 | Miscible | 1 | 2 | 1 | | | | | 1 | | |
| | ACMO | −1.08 | 100 | | | | 1 | | | | | | |
| | DEAA | −0.77 | Soluble | | | | | 1 | | | | | |
| | DMAPAA | 14.88 | Soluble | | | | | | | | | 1 | |
| | NIPAM | 14.83 | <0.1 | | | | | | | | | | 1 |
| | KBM-503 | — | — | 0.05 | 0.05 | 0.025 | 0.025 | 0.025 | | 0.025 | 0.025 | 0.05 | 0.025 |

| | | | | | | | | | | | | |
|---|---|---|---|---|---|---|---|---|---|---|---|---|
| Chain transfer agent | — | — | 0.05 | 0.05 | 0.05 | 0.05 | 0.025 | 0.025 | 0.025 | 0.025 | 0.05 | 0.05 |
| Emulsifier A | — | — | 2 | 2 | 2 | 2 | 2 | 2 | | 2 | 2 | 2 |
| Emulsifier B | — | — | | | | | | | 4 | | | |

TABLE 2

| | | | | | | | Comp. | Comp. | Comp. | Comp. | Comp. |
|---|---|---|---|---|---|---|---|---|---|---|---|
| | | Ex. 1 | Ex. 2 | Ex. 3 | Ex. 4 | Ex. 5 | Ex. 1 | Ex. 2 | Ex. 3 | Ex. 4 | Ex. 5 |
| Adhesive | on Bakelite plate | 3.8 | 4.0 | 3.6 | 3.7 | 3.7 | 0.6 | 2.7 | 2.9 | 3.1 | 3.3 |
| strength (N/10 mm) | on acrylic plate | 4.7 | 5.2 | 4.8 | 5.0 | 4.8 | 2.4 | 3.3 | 3.5 | 3.4 | 4.3 |
| | Water-resistant adhesive strength | 2.5 | 2.1 | 1.9 | 1.8 | 1.2 | 0.4 | 0.8 | 0.4 | 0.7 | 0.8 |
| On-skin adhesive strength (N/50 mm) | | 5.6 | 4.5 | 4.9 | 5.1 | 4.2 | 3.2 | 1.2 | 4.3 | 3.9 | 4.2 |
| Sweat-resistant adhesive strength (N/50 mm) | | 3.6 | 2.8 | 3.1 | 3.3 | 2.6 | 0.6 | 0.7 | 1.2 | 0.5 | 0.3 |
| Storage modulus | 25° C. | 0.49 | 0.65 | 0.48 | 0.50 | 0.48 | 0.38 | 0.80 | 0.47 | 0.48 | 0.47 |
| G′ (MPa) | 37° C. | 0.36 | 0.48 | 0.35 | 0.36 | 0.35 | 0.30 | 0.65 | 0.35 | 0.36 | 0.36 |

| | | | | | | | | | | |
|---|---|---|---|---|---|---|---|---|---|---|
| Tan δ peak-top temperature (° C.) | −40 | −36 | −41 | −39 | −42 | −35 | −30 | −43 | −41 | −42 |

As shown in Tables 1 and 2, the PSAs according to Examples 1 to 5 (using a monomer composition that included both a carboxy group-containing monomer and an N-containing but NH-free monomer, wherein AA and a monomer more hydrophobic than AA are used as the carboxy group-containing monomer) exhibited superior water resistance and high adhesive strength on both Bakelite and acrylic plates in comparison with Comparative Examples 1 to 5 not satisfying the monomer composition. The on-skin adhesive strength was also high, maintaining high sweat-resistant adhesive strength in particular. Among them, Examples 1 to 4 using an N-heterocyclic monomer as the N-containing monomer were found to have greater adhesive strength and water resistance. It is noted that the PSAs according to Examples 1 to 5 all had good flexibility, having a storage modulus G′ of less than 1 MPa whether at 25° C. or at 37° C. and also a tan δ (G″/G′) peak-top temperature below −30° C.

On the other hand, Comparative Example 1 showed low adhesive strength and inferior water resistance, without combined use of a carboxy group-containing monomer and an N-containing monomer and with only AA used as the carboxy group-containing monomer. With respect to Comparative Example 3 using just AA as the carboxy group-containing monomer with an N-containing but NH-free monomer, the adhesive strength improved as compared with Comparative Example 1, but did not reach the level of Examples 1 to 5; and the water resistance did not improve. This indicates that the combined use of a carboxy group-containing monomer and an N-containing monomer is not sufficient to obtain high adhesive strength and water resistance. As for Comparative Example 2 using both AA and MAA as carboxy group-containing monomers with no N-containing monomer was used, the adhesive strength obtained was not at a comparable level to the working examples, and the water resistance did not improve much, either. The PSA of Comparative Example 2 had low flexibility, showing especially low adhesive strength on the skin. With respect to Comparative Examples 4 and 5 using both AA and MAA as carboxy group-containing monomers along with an NH-containing monomer (DMAPAA, NIPAM), relatively high adhesive strength was obtained, but was not as high as Examples 1 to 5, and the water resistance and sweat resistance were also low. These results show that the combined use of carboxy group-containing monomers and a N-containing but NH-free monomer contributes greatly to improved water resistance and sweat resistance.

The results discussed above indicates that it is possible to obtain a PSA having high adhesive strength and good water resistance even when designed to be flexible according to a water-dispersed PSA composition that comprises an acrylic polymer, with the acrylic polymer including, as a monomer mixture, an alkyl (meth)acrylate (m1) as the primary component and further including a carboxy group-containing monomer (m2) and an N-containing but NH-free monomer (m3) wherein the carboxy group-containing monomer (m2) includes AA and a more-hydrophobic-than-AA monomer. When using such a PSA as dermal PSA, it can tightly adhere to the skin and show excellent sweat resistance.

Although specific embodiments of the present invention have been described in detail above, these are merely for illustrations and do not limit the scope of claims. The art according to the claims includes various modifications and changes made to the specific embodiments illustrated above.

### REFERENCE SIGNS LIST

1 PSA sheet
21 PSA layer
21A first adhesive face
21B second adhesive face
31, 32 release liners

1. A water-dispersed pressure-sensitive adhesive composition comprising an acrylic polymer, wherein
the acrylic polymer is a polymer of a monomer mixture that comprises an alkyl (meth)acrylate (m1) as a primary component and further comprises a carboxy group-containing monomer (m2) and a nitrogen-atom-containing monomer (m3) with no hydrogen atoms bonded to the nitrogen atom, and
the carboxy group-containing monomer (m2) comprises acrylic acid (m2a) and a monomer (m2b) that is more hydrophobic than acrylic acid (m2a).

2. The water-dispersed pressure-sensitive adhesive composition according to claim 1, wherein the nitrogen-atom-containing monomer (m3) has an acid dissociation constant (pKa) in the range of −2 or higher and 1 or lower.

3. The water-dispersed pressure-sensitive adhesive composition according to claim 1, wherein the nitrogen-atom-containing monomer (m3) has a cyclic structure with a cyclic nitrogen atom.

4. The pressure-sensitive adhesive composition according to claim 1, wherein the monomer (m2b) more hydrophobic than acrylic acid has a water solubility in the range of 2.0 g/100 mL to 15.0 g/100 mL at 20° C.

5. The water-dispersed pressure-sensitive adhesive composition according to claim 1, wherein the monomer mixture contains the carboxy group-containing monomer (m2) in an amount of 0.5% by weight or more and 5% by weight or less, and the nitrogen-atom-containing monomer (m3) in an amount of 0.1% by weight or more and 5% by weight or less.

6. The water-dispersed pressure-sensitive adhesive composition according to claim 1, wherein the monomer mixture further comprises a silane-based monomer.

7. A pressure-sensitive adhesive sheet that has a pressure-sensitive adhesive layer formed from the water-dispersed pressure-sensitive adhesive composition according to claim 1.

8. The pressure-sensitive adhesive sheet according to claim 7, wherein the pressure-sensitive adhesive sheet is used on the skin.
